(19) United States
(12) Patent Application Publication
(10) Pub. No.: US 2020/0247841 A1
(43) Pub. Date: Aug. 6, 2020

(54) SYNTHESIS OF ICATIBANT

(71) Applicant: BIOCON LIMITED, Bangalore (IN)

(72) Inventors: Vasanthakumar Ganga RAMU, Bengaluru (IN); Nitin Sopanrao PATIL, Bengaluru (IN); Ventaka Raghavendracharyul PALLE, Hyderabad (IN); YOGESHA, Magadi Town (IN)

(21) Appl. No.: 16/651,396

(22) PCT Filed: Sep. 27, 2018
(86) PCT No.: PCT/IB2018/057498

§ 371 Date: Mar. 27, 2020

(30) Foreign Application Priority Data
Sep. 27, 2017 (IN) 201741034314

(51) Int. Cl. C07K 1/06; C07K 1/14; C07K 1/10; C07K 7/06

(52) U.S. Cl. CPC C07K 1/061; C07K 7/06; C07K 1/10; C07K 1/14

(57) ABSTRACT

The present invention relates to the efficient solid-phase synthesis of Icatibant represented by Formula (I). The present invention relates to an efficient process for the preparation of Icatibant by sequential coupling employing solid phase approach. It involves sequential coupling of protected amino acids to prepare Icatibant. The present invention also involves the usage of inorganic salts during the coupling, wash with HOBt in DMF solution after Fmoc-deprotection step to ensure complete removal of piperidine and reactions are going for completion, and thus avoid addition/deletion sequences and also improve the process yield.

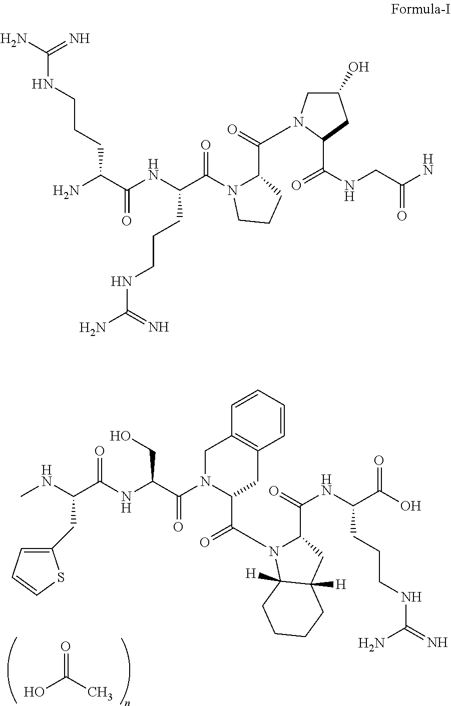

### TECHNICAL FIELD OF THE INVENTION

The present invention relates to the efficient solid-phase synthesis of Icatibant represented by Formula-I.

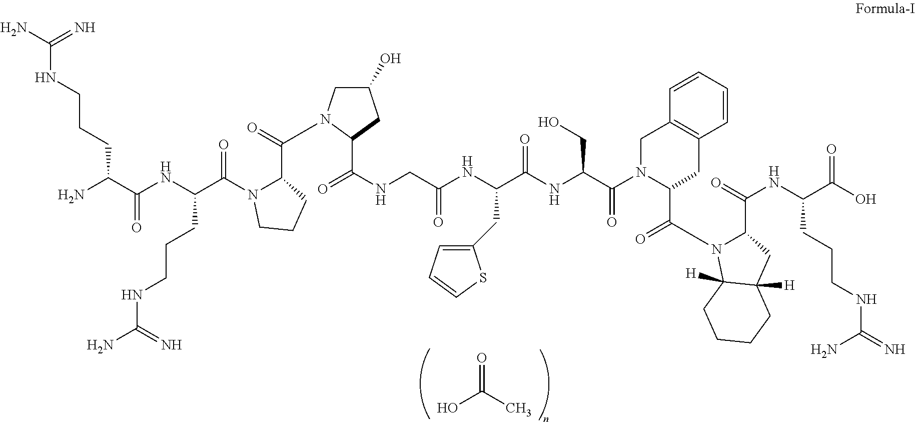

Icatibant (FIRAZYR®) is indicated for the treatment of acute attacks of hereditary angioedema (HAE) in adults 18 years of age and older.

### BACKGROUND OF THE INVENTION

Icatibant is a competitive antagonist selective for the bradykinin B2 receptor, with an affinity similar to bradykinin. Hereditary angioedema is caused by an absence or dysfunction of C1-esterase-inhibitor, a key regulator of the Factor XII/kallikrein proteolytic cascade that leads to bradykinin production. Bradykinin is a vasodilator which is thought to be responsible for the characteristic HAE symptoms of localized swelling, inflammation, and pain. Icatibant inhibits bradykinin from binding the B2 receptor and thereby treats the clinical symptoms of an acute, episodic attack of HAE. FIRAZYR® (icatibant) is a synthetic decapeptide with five non-proteinogenic amino acids.

Icatibant (FIRAZYR®) developed by Shire Orphan Therapies got initial approval in United States in 2011 as subcutaneous injection.

U.S. Pat. No. 5,648,333 B1 discloses Icatibant and process for preparing it.

CN102532267 discloses process for the preparation of Icatibant using CTC resin using different coupling agents for coupling different amino acids.

CN103992383 discloses a process for the preparation of Icatibant using liquid phase synthesis of Boc-D-Arg-Arg-OH.2HCl followed by coupling with the remaining fragment by solid phase synthesis using Wang resin.

CN104072585 discloses a process for the preparation of Icatibant using sequential coupling of amino acids with Wang resin or a p-hydroxymethylphenoxymethylstyrene resin as solid support.

WO2016157177 discloses a process for the preparation of Icatibant in presence of the biologically compatible tertiary amine nicotinamide as catalyst.

5473/CHE/2014 discloses a process for the preparation of Icatibant using sequential coupling of amino acids on wang resin.

The prior processes for preparing Icatibant have disadvantages. The methods are not suitable for large scale production of Icatibant due to complex techniques and high costs; hence, the processes are not commercially viable or execution problems persist.

During solid phase synthesis, it is observed that sturdy tendency of peptides to aggregate under conditions employed. It is because of the following reasons:

Growing peptide sequence is susceptible to form β-sheet kind of structures, which results in collapse of the peptidyl resin; in such conditions, the dispersion of reagents into the peptidyl resin is limited, coupling and deprotection reactions will be sluggish and incomplete, thereby generating deletion/addition, racemization peptide impurities leading to difficulties in purification and resulting in low yield. Hence, there remains a need to provide efficient process for preparation of Icatibant which is high yielding, scalable, cost effective, environment friendly and commercially viable by avoiding repeated cumbersome and lengthy purification steps.

### OBJECTS OF THE INVENTION

The objective of the present invention is to develop simple, robust, and commercially viable sequential process for the preparation of Icatibant of the Formula I with the aid of inorganic salts, novel and efficient coupling conditions, deprotection and washing conditions after each amino acid in the sequence.

### BRIEF DESCRIPTION OF DRAWINGS

FIG. 1: Flow chart for process for solid-phase synthesis of Icatibant (Formula-I) according to the present invention.

### SUMMARY OF THE INVENTION

An embodiment of the present invention involves synthetic process for the preparation of Icatibant comprising the following steps:
A) Loading of Arginine to a resin solid-phase support in the presence of coupling agent.
B) Capping of the unreacted functional sites.
C) Sequential coupling of side chain protected amino acids to prepare backbone of Icatibant, in the presence of coupling agent and an inorganic salt.
D) Crude Icatibant is obtained by removal of protective groups and cleavage of peptide from the resin.
E) Purification of crude Icatibant.

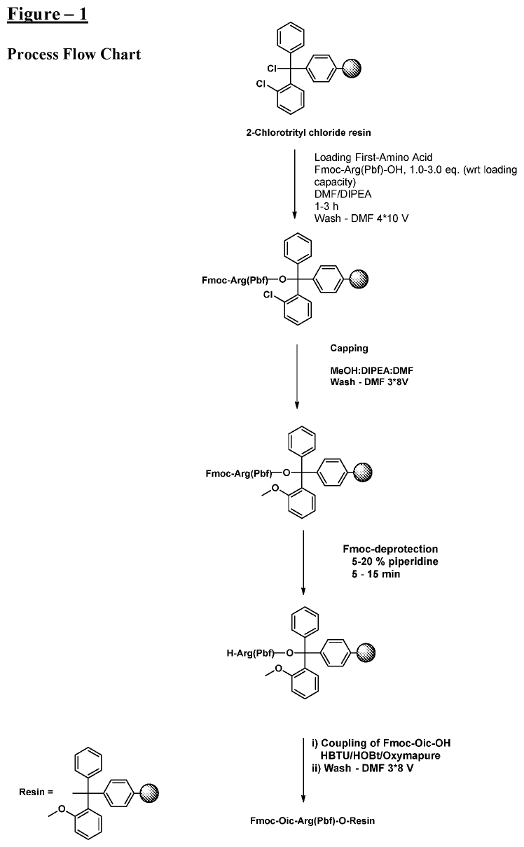

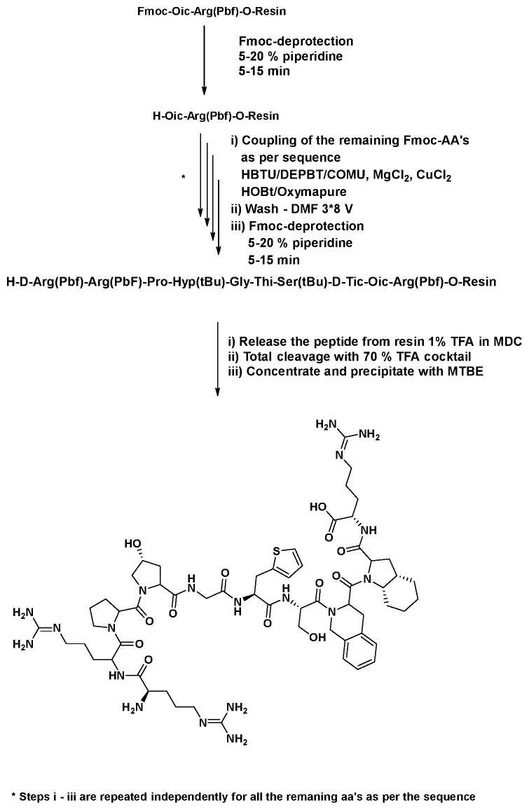

The schematic description of the process is as shown in FIG. 1

The approach employed is solid phase peptide synthesis of Icatibant by sequential approach and involves inorganic salts during coupling along with regular coupling agents and additives. The method offers completion of coupling and deprotection reactions and reduction in racemization and thereby control the isomeric impurities which are very close to the target molecule and in turn ease the purification process of the peptide.

### DETAILED DESCRIPTION OF THE INVENTION

The present invention relates to an efficient process for the preparation of Icatibant by sequential coupling of individual amino acids by employing solid phase approach. The approach employed at the present invention is solid phase manual peptide synthesis by using 2-chlorotrityl chloride as solid support, Fmoc-/tBu approach and includes site specific efficient coupling agents and inorganic salts used during coupling along with regular coupling agents and additives. And after deprotection of Fmoc-group using 15-20% piperidine the peptidyl resin was washed with 0.01-0.5 M HOBt/DMF instead of plain DMF to ensure complete removal of piperidine which was advantageous to avoid insertion impurities. The complete synthesis is achieved through sequential approach. The method offers completion of coupling and deprotection reactions and reduction in racemization and thereby control the isomeric impurities which are very close to the target molecule and in turn ease the purification process of the peptide.

The invention is represented by following examples. These examples are for illustration only and hence should not be construed as limitation of the scope of invention.

### Abbreviations:

### ACN: Acetonitrile

Boc: tert-Butyloxycarbonyl
COMU: 1-Cyano-2-ethoxy-2-oxoethylidenaminooxy)dimethylamino-morpholino-carbenium hexafluorophosphate
CuCl₂: Copper chloride
DEPBT: 3-(Diethoxyphosphoryloxy)-1,2,3-benzotriazin-4(3H)-one
DIC: NN′-diisopropylcarbodiimide
DMAP: Dimethylamino pyridine

### DMF: N,N′-Dimethylformamide

### DIPEA: Diisopropylethylamine

Fmoc: 9-fluorenylmethoxycarbonyl
HBTU: O-Benzotriazole-N,N,N′,N′-tetramethyl uronium hexafluorophosphate

### HOBt: N-Hydroxybenzotriazole

HFIP: 1,1,1,3,3,3-Hexafluro-2-propanol

### Mtt: Methyltrityl

### MeOH: Methanol

### MgCl₂: Magnesium Chloride

### NMM: N-methylmorpholine

NMP: N-Methyl-2-pyrrolidone

### TES: Triethylsilane

### TFE: Trifluroethanol

TFA: Trifluoroacetic acid

### Pbf: 2,2,4,6,7-Pentamethyldihydrobenzofurane

RT: Room temperature
^{t}Bu: tert-Butyl

### TIS: Triisopropyl Silane

### Trt: Trityl

### TMP: 2,4,6-Trimethylpyridine

The invention is represented by following examples. These examples are for illustration only and hence should not be construed as limitation of the scope of invention.

### Example 1

CTC resin (10 g) with substitution 1.22 mmol/g was taken in solid phase peptide synthesis vessel. Swelling of the resin was carried out in DMF (80-100 mL) for 1 h and drained. Fmoc-Arg(Pbf)-OH (7.9 g, 1.0 eq.) was dissolved in DMF (80-100 mL) and added to swollen resin. DIPEA, NMM, and TMP (2.0-4.0 eq.), preferably DIPEA was added under stirring. Continued stirring for 2-3 h at room temperature and the reaction mixture was drained. Washed the resin with DMF (80-100 mL). First amino acid loading estimation was performed and it was found to be 0.61 mmol/g. Capping of the unreacted sites of the resin was carried out using Methanol (10%), DIPEA (4%) in DMF (86 mL). Washed the resin with DMF (80-100 mL*3). Deblocking of Fmoc was carried out by treating the resin with 5-20 piperidine in DMF twice for the period of 5-15 min. Washed the resin with 0.01-0.5 M HOBt solution in DMF (100 mL*2) followed by DMF (80-100 mL*2).

Fmoc-Oic-OH (2.0-4.0 eq.) and HOBt (2.0-4.0 eq.) was dissolved in DMF (80-100 mL) and added to the reaction vessel. Stirred the reaction mass and added HBTU (2.0-4.0 eq.) dissolved in DMF (20 mL). Added DIPEA (4.0-6.0 eq) and stirred the mass under nitrogen atmosphere for 1.0-2.0 h. Monitored the completion of coupling through Kaiser test. After completion of coupling, the resin was washed with DMF (80-100 mL*3) and taken for deblocking of Fmoc. Rest of the amino acids couplings in the sequence were carried out using HBTU/HOBt. After coupling of all the amino acids as per the sequence deprotection of Fmoc was carried out and the peptidyl resin was washed with DMF, DCM, Methanol and MTBE. Each wash was performed twice for the period of 3 min. The peptidyl resin was dried and taken for total cleavage. Peptidyl resin (30 g) was treated with TFA:TIS:Water in the ratio of 90:5:5 at room temperature. Stirred for 3 h, filtered and the filtrate was concentrated to half of its volume and isolated the solid using MTBE. Crude peptide (14.4 g) isolated has purity of 68.69%. 0.88 RRT impurity was 3.55%, 0.97 RRT impurity was 1.46% and 1.09 RRT impurity was 0.15%.

### Example 2

Synthesis of the peptide was carried out similar to Example 1 but all the couplings and deprotections were performed at around 38° C. Total cleavage was also performed similar to example 1, resulting in crude peptide with purity 67.08%. 0.88 RRT impurity was 4.51%, 0.97 RRT impurity was 2.61% and 1.09 RRT impurity was 0.80%.

### Example 3

Synthesis of the peptide was carried out similar to Example 1. Coupling reactions of amino acids particularly Fmoc-Oic-OH, Fmoc-D-Tic-OH, Fmoc-Thi-OH, Fmoc-Gly-OH, Fmoc-Hyp(tBu)-OH, Fmoc-Pro-OH were carried out using HBTU/HOBt in presence of DIPEA and catalytic amount of Copper(II)Chloride/Magnesium chloride. And, Fmoc-Ser(tBu)-OH, Fmoc-Arg(Pbf)-OH and Fmoc-D-Arg(Pbf)-OH coupling reactions performed using DEPBT/Oxymapure in the presence of DIPEA, NMM, TMP, preferably DIPEA and catalytic amount of Copper(II) Chloride/Magnesium chloride. After the coupling of all the amino acids according to sequence, deblocking of Fmoc-group was carried out and the peptidyl resin was washed with DMF, DCM, Methanol and MTBE. Each wash was performed twice for the period of 3 min. The peptidyl resin was dried and taken for total cleavage. Peptidyl resin (30 g) was treated with TFA:TIS:Water in the ratio of 90:5:5 at room temperature. Stirred for 3 h, filtered and the filtrate was concentrated to half of its volume and isolated the solid using MTBE and dried in vacuo. Purity: 72.38%, 0.88 RRT impurity was 0.26%.

### Example 4

Synthesis of the peptide was carried out similar to Example 3. Upon completion of the sequence, the protected peptide was released from resin using 1 TFA in MDC. Peptidyl resin (12 g) was treated with 1% TFA in MDC (120 mL*12 washes). Each wash was carried out for the period of 5 min. The combined washings were pooled and concentrated to dryness. The obtained residue was treated with TFA:MDC:Phenol:m-cresol:TIS cocktail (120 mL) in the ratio of 70:10:10:5:5 for 3 h at room temperature. Crude peptide (5.6 g) was isolated using MTBE. Purity was around 77.92%, 0.88 RRT impurity was 0.17%.

### Example 5

Synthesis of the peptide was carried out similar to Example 1. Coupling reactions of amino acids particularly Fmoc-Oic-OH, Fmoc-D-Tic-OH, Fmoc-Thi-OH, Fmoc-Gly-OH, Fmoc-Hyp(tBu)-OH, Fmoc-Pro-OH, Fmoc-Arg(Pbf)-OH, and Fmoc-D-Arg(Pbf)-OH were carried out using HBTU/HOBt in presence of DIPEA and catalytic amount of Copper(II)Chloride/Magnesium chloride. And, Fmoc-Ser(tBu)-OH coupling was performed using DIC/HOBt in the presence of DIPEA, NMM, TMP, preferably DIPEA and catalytic amount of Copper(II) Chloride/Magnesium chloride. After the coupling of all the amino acids according to sequence, deblocking of Fmoc-group was carried out and the peptidyl resin was washed with DMF, DCM, Methanol and MTBE. Each wash was performed twice for the period of 3 min. The peptidyl resin was dried and taken for cleavage using 1 TFA in MDC. Peptidyl resin (12 g) was treated with 1% TFA in MDC (120 mL*12 washes). Each wash was carried out for the period of 5 min. The combined filtrate was concentrated to dryness and treated with TFA: MDC: Phenol:m-cresol:TIS cocktail (120 mL) in the ratio of 70:10:10:5:5 for 3 h at room temperature. Crude peptide was isolated using MTBE. Purity: 64.97%, 0.88 RRT impurity was 0.97%.

### Example 6

Synthesis of the peptide was carried out similar to Example 1. Coupling reactions of amino acids particularly Fmoc-Oic-OH, Fmoc-D-Tic-OH, Fmoc-Thi-OH, Fmoc-Gly-OH, Fmoc-Hyp(tBu)-OH, Fmoc-Pro-OH, Fmoc-Arg(Pbf)-OH, and Fmoc-D-Arg(Pbf)-OH were carried out using HBTU/HOBt in presence of DIPEA and catalytic amount of Copper(II)Chloride/Magnesium chloride. And, Fmoc-Ser(tBu)-OH coupling was performed using HATU/HOBt in the presence of DIPEA, NMM, TMP, preferably DIPEA and catalytic amount of Copper(II) Chloride/Magnesium chloride. After the coupling of all the amino acids according to sequence, deblocking of Fmoc-group was carried out and the peptidyl resin was washed with DMF, DCM, Methanol and MTBE. Each wash was performed twice for the period of 3 min. The peptidyl resin was dried and taken for cleavage using 1% TFA in MDC. Peptidyl resin (12 g) was treated with 1% TFA in MDC (120 mL*12 washes). Each wash was carried out for the period of 5 min. The combined filtrate was concentrated to dryness and treated with TFA: MDC: Phenol:m-cresol:TIS cocktail (120 ML) in the ratio of 70:10:10:5:5 for 3 h at room temperature. Crude peptide was isolated using MTBE. Purity: 74.54%, 0.88 RRT impurity was 1.55%.

### Example 7

Synthesis of the peptide was carried out similar to Example 1. Coupling reactions of amino acids particularly Fmoc-Oic-OH, Fmoc-D-Tic-OH, Fmoc-Thi-OH, Fmoc-Gly-OH, Fmoc-Hyp(tBu)-OH, Fmoc-Pro-OH, Fmoc-Arg(Pbf)-OH, and Fmoc-D-Arg(Pbf)-OH were carried out using HIBTU/HOBt/Oxymapure in presence of DIPEA and catalytic amount of Copper(II)Chloride/Magnesium chloride. And, Fmoc-Ser(tBu)-OH coupling was performed using PyBOP/HOBt/Oxymapure in the presence of DIPEA, NMM, TMP, preferably DIPEA and catalytic amount of Copper(II) Chloride/Magnesium chloride. After the coupling of all the amino acids according to sequence, deblocking of Fmoc-group was carried out and the peptidyl resin was washed with DMF, DCM, Methanol and MTBE. Each wash was performed twice for the period of 3 min. The peptidyl resin was dried and taken for cleavage using 1 TFA in MDC. Peptidyl resin (12 g) was treated with 1 TFA in MDC (120 mL*12 washes). Each wash was carried out for the period of 5 min. The combined filtrate was concentrated to dryness and treated with TFA:MDC:Phenol:m-cresol: TIS cocktail (120 ML) in the ratio of 70:10:10:5:5 for 3 h at room temperature. Crude peptide was isolated using MTBE.

### Example 8

Synthesis of the peptide was carried out similar to Example 1. Coupling reactions of amino acids particularly Fmoc-Oic-OH, Fmoc-D-Tic-OH, Fmoc-Thi-OH, Fmoc-Gly-OH, Fmoc-Hyp(tBu)-OH, Fmoc-Pro-OH, Fmoc-Arg(Pbf)-OH, and Fmoc-D-Arg(Pbf)-OH were carried out using HBTU/HOBt in presence of DIPEA and catalytic amount of Copper(II)Chloride/Magnesium chloride. And, Fmoc-Ser(tBu)-OH coupling was performed using COMU/Oxymapure in the presence of DIPEA, NMM, TMP, preferably DIPEA and catalytic amount of Copper(II) Chloride/Magnesium chloride. After the coupling of all the amino acids according to sequence, deblocking of Fmoc-group was carried out and the peptidyl resin was washed with DMF, DCM, Methanol and MTBE. Each wash was performed twice for the period of 3 min. The peptidyl resin was dried and taken for cleavage using 1% TFA in MDC. Peptidyl resin (12 g) was treated with 1% TFA in MDC (120 mL*12 washes). Each wash was carried out for the period of 5 min. The combined filtrate was concentrated to dryness and treated with TFA:MDC:Phenol:m-cresol:TIS cocktail (120 ML) in the ratio of 70:10:10:5:5 for 3 h at room temperature. Crude peptide was isolated using MTBE.

### Example 9

Synthesis of the peptide was carried out similar to Example 1, with 35 mmol scale. Coupling of all the amino acids were carried out using HBTU/Oxymapure in presence of DIPEA and catalytic amount of Copper (II) Chloride/Magnesium chloride and upon completing the sequence the peptidyl resin was washed with DMF, DCM, Methanol and MTBE, peptidyl resin was dried in vacuo and released the protected peptide using 1% TFA in MDC. Peptidyl resin (130 g) was treated with 1% TFA in MDC (1.3 L*15 washes). Each wash was carried out for the period of 5 min. The combined filtrate was concentrated to dryness and total cleavage of the peptide was carried out by treating with TFA:MDC:Phenol:m-cresol:TIS:H₂O cocktail in the ratio of 70:10:5:5:5:5 for 3 h at room temperature. Crude peptide was isolated using MTBE with a purity of 76.8% and 68% yield.

1. A process for the preparation of Icatibant comprising the steps of,
a) Loading of Arginine (Pbf)-OH to a resin solid-phase support,
b) Capping of the unreacted functional sites,
c) Sequential coupling of side chain protected amino acids to prepare Icatibant, in the presence of coupling agent and an inorganic salt,
d) Crude Icatibant is obtained by removal of protective groups and cleavage of peptide from the resin, and
e) Optionally purifying crude Icatibant.

2. The process for the preparation of Icatibant of claim 1, comprising the steps of,
a) Loading of Arginine (Pbf) to a resin solid-phase support in the presence of coupling agent,
b) Sequential coupling of side chain protected amino acids to prepare Icatibant, in the presence of coupling agent, oxymapure and an inorganic salt,
c) Crude Icatibant is obtained by removal of protective groups and cleavage of peptide from the resin, and
d) Optionally purifying crude Icatibant.

3. The process for the preparation of Icatibant of claim 1, wherein the coupling agent is selected from the group consisting of HBTU, COMU, DEPBT, and any combination thereof.

4. The process for the preparation of Icatibant of claim 1, wherein the coupling additives selected from oxymapure, HOBt or any combination thereof.

5. The process for the preparation of Icatibant of claim 1, wherein the deprotection of protecting groups from step d is followed by washing with 0.01-0.5 M HOBt in DMF and total cleavage from the resin using TFA:MDC:Phenol:m-cresol:TIS:H₂O.
